(19) United States
(12) Patent Application Publication
(10) Pub. No.: US 2026/0277988 A1
(43) Pub. Date: Sep. 17, 2026

(54) INFORMATION DISPLAY FOR PATIENTS

(71) Applicant: Thomas Whealton, Lexington, KY (US)

(72) Inventor: Thomas Whealton, Lexington, KY (US)

(21) Appl. No.: 19/567,433

(22) Filed: Mar. 16, 2026

Related U.S. Application Data
(60) Provisional application No. 63/772,079, filed on Mar. 14, 2025.

(51) Int. Cl. G06F 16/435; G06F 3/14; G06F 16/438; G16H 40/67; G16H 50/20; H04N 21/214; H04N 21/239; H04N 21/262

(52) U.S. Cl. CPC G06F 16/435; G06F 3/14; G06F 16/438; G16H 40/67; G16H 50/20; H04N 21/2143; H04N 21/2396; H04N 21/26258

(57) ABSTRACT

A system for automated information display may include at least one display associated with a room. The system may also include a content database including digital content. The digital content may include one or more videos and descriptions related to a procedure. The system may further include a subscriber system that may be operable to access a subset of the digital content in the content database. The subscriber system may also be operable to obtain first digital content from the content database based on a first profile associated with a first patient. The subscriber system may further be operable to direct the first digital content to be displayed on the at least one display corresponding to the room where the first patient may be located.

### CROSS REFERENCE TO RELATED APPLICATIONS

This U.S. Patent Application claims priority to U.S. Provisional Patent Application No. 63/772,079, titled “AUTOMATED INFORMATION DISPLAY,” and filed on Mar. 14, 2025, the disclosure of which is hereby incorporated by reference in its entirety.

### TECHNICAL FIELD

This disclosure relates to displaying information, and more specifically, to an automated information display for patients.

### BACKGROUND

Unless otherwise indicated herein, the materials described herein are not prior art to the claims in the present application and are not admitted to be prior art by inclusion in this section.

Patients waiting to undergo surgery or other medical procedures may have questions about preparations for the surgery/procedure, the performance of the procedure, and/or the post-procedure process. In some instances, patients may not have knowledge about the procedure to know what questions to ask or what to anticipate. Further, it may be beneficial to a clinic to quickly and/or automatically distribute information to patients based on a diagnosis given to a patient.

The subject matter claimed in the present disclosure is not limited to implementations that solve any disadvantages or that operate only in environments such as those described above. Rather, this background is only provided to illustrate one example technology area where some embodiments described in the present disclosure may be practiced.

### SUMMARY

In an example embodiment, a method may include requesting access to a content database. The content database may include one or more digital content. The method may also include obtaining first digital content and second digital content of the one or more digital content from the content database. The method may further include providing the first digital content to a first room. The method may also include providing the second digital content to a second room.

In another embodiment, a system for automated information display may include at least one display associated with a room. The system may also include a content database including digital content. The digital content may include one or more videos and descriptions related to a procedure. The system may further include a subscriber system that may be operable to access a subset of the digital content in the content database. The subscriber system may also be operable to obtain first digital content from the content database based on a first profile associated with a first patient. The subscriber system may further be operable to direct the first digital content to be displayed on the at least one display corresponding to the room where the first patient may be located.

The objects and advantages of the embodiments will be realized and achieved at least by the elements, features, and combinations particularly pointed out in the claims.

Both the foregoing general description and the following detailed description are given as examples and are explanatory and not restrictive of the invention, as claimed.

### DESCRIPTION OF DRAWINGS

Example implementations will be described and explained with additional specificity and detail using the accompanying drawings in which:

FIG. 1 illustrates a block diagram of an example system for automated information display for patients;

FIG. 2 illustrates an example user interface for information display for a patient;

FIG. 3 illustrates an example user interface for an interactive information display for a patient;

FIG. 4 illustrates a flowchart of an example method of automated information display for patients; and

FIG. 5 illustrates an example computing device.

### DETAILED DESCRIPTION

In instances in which patients seek medical professionals for various procedures, the patients may lack an understanding of the procedure, information related to pre-procedure expectations, and/or information related to post-procedure expectations. In some instances, the patients may lack the understanding associated with a particular procedure such that the patients may be unaware of potential concerns, and/or questions to ask a medical professional.

In some instances, providing procedure information to patients may help the patients uncover additional questions or concerns, feel more confident about the procedure, and or have a better experience throughout the procedure. In some instances, automatically providing information to the patients related to a particular procedure to be performed may provide information to the patients such that the patients may be better informed and/or better prepared for the particular procedure.

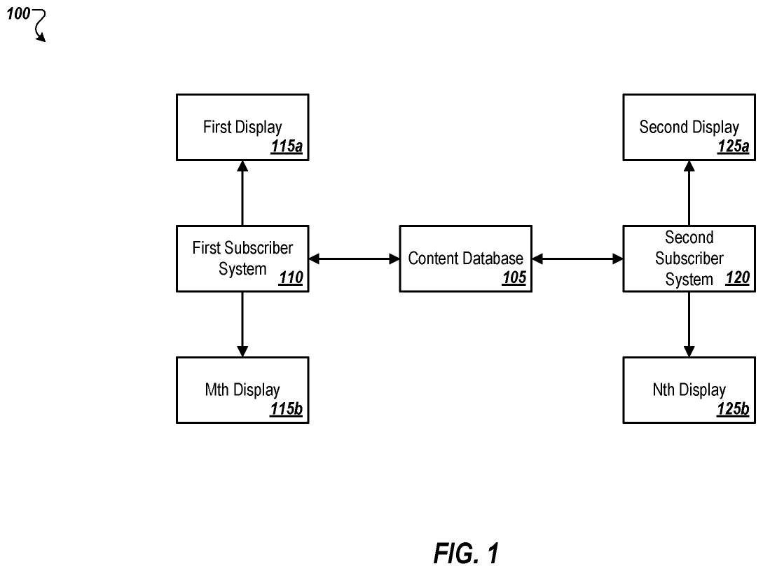

FIG. 1 illustrates a block diagram of an example system 100 for automated information display for patients. The system 100 may include a content database 105, a first subscriber system 110 and a second subscriber system 120. The first subscriber system 110 may have access to control content displayed on a first display 115a, and/or an mth display 115b. The second subscriber system 120 may have access to control content displayed on a second display 125a and/or an nth display 125b.

In some instances, the content database 105 may include a collection of digital content that may be distributed to at least the first subscriber system 110 and/or the second subscriber system 120. In some instances, the digital content in the content database 105 may include one or more videos and/or descriptions related to a procedure, such as a medical procedure. For example, the digital content in the content database 105 may include at least videos related to a first diagnosis, treatment of a condition associated with the first diagnosis, results of the treatment of the condition, and follow-up procedures associated with the treatment.

In some instances, the digital content may be provided to a particular patient via the first display 115 upon the particular patient entering a room, such as a waiting room, a private room, and/or other similar locations where a patient may receive private care. For example, the particular patient may enter the room and after a discussion with a nurse, assistant, or other personnel (e.g., not a doctor), the digital content associated with a preliminary diagnosis, a potential diagnosis, and/or information associated with the particular patient may be displayed on the first display 115a prior to meeting with the doctor. In some instances, the digital content provided to the particular patient may be based on a profile associated with the particular patient. Using the previous example, the profile associated with the particular patient may include the preliminary diagnosis, the potential diagnosis, and/or information associated with the particular patient

Alternatively, or additionally, the profile associated with the particular patient may include responses associated with intake questions and/or symptoms experienced by the particular patient. As such, digital content may be displayed to the particular patient based on the responses to intake questions and/or the symptoms experienced by the particular patient, prior to the particular patient entering the room. For example, the particular patient may arrive at a location associated with the first subscriber system 110 and may provide symptoms to an intake person and/or intake device. In response to the intake responses, digital content may be displayed, which may include digital content associated with a potential diagnosis, anticipated treatment and/or care associated with the symptoms and/or the potential diagnosis, and so forth.

In some instances, the digital content included in the content database 105 may be arranged in one or more playlists, which may correspond to a particular diagnosis. For example, a first playlist may correspond with a first diagnosis, a second playlist may correspond with a second diagnosis, and so forth. Alternatively, or additionally, the playlists may be curated by a user within the first subscriber system 110 and/or the second subscriber system 120. For example, a doctor using the first subscriber system 110 may determine that from a subset of the digital content associated with a particular condition, some digital content may be included and some digital content may be excluded, where the result thereof may be curated into a playlist for the particular condition. In such instances, the curated playlist may differ from a playlist obtained from the content database 105 that may be associated with the same or similar as the particular condition.

Alternatively, or additionally, the digital content in the content database 105 may include multiple videos that may be selected on an individual basis, such as based on a need of the first subscriber system 110 and/or the second subscriber system 120. For example, in instances in which a particular patient receives a particular diagnosis that does not have a corresponding digital content accessible to the first subscriber system 110 (and/or the second subscriber system 120), the first subscriber system 110 (and/or the second subscriber system 120) may request additional digital content from the content database 105 corresponding to the particular diagnosis.

In some instances, the particular diagnosis may not have corresponding digital content in the content database 105. In such instances, when the particular diagnosis is received at the content database 105, the content database 105 may generate an indication that one or more digital content may be created and/or added to the content database 105 to correspond with the particular diagnosis. In some instances, the indication may be automatically generated and/or may be obtained by a manager of the content database 105, where the manager may be operable to create one or more additional digital content for the content database 105.

In some instances, access to the content database 105 may be restricted such that the first subscriber system 110 and/or the second subscriber system 120 may have access to a subset of the digital content in the content database 105. For example, the first subscriber system 110 may have access to a first subset of the digital content in the content database 105 and the second subscriber system 120 may have access to a second subset of the digital content in the content database 105. The first subset and the second subset may include digital content that may be substantially the same, the first subset and the second subset may have some overlapping digital content, and/or the first subset and the second subset may be different from one another. In some instances, as new digital content may be added to the content database 105 (such as in response to digital content not being available to the first subscriber system 110 and an indication is generated and/or provided to the content database 105 (e.g., to a manager of the content database 105)), the subscribers (e.g., the first subscriber system 110 and/or the second subscriber system 120) may add the new digital content to their corresponding subset.

In some instances, the first subscriber system 110 and/or the second subscriber system 120 may form an agreement with an owner of the content database 105 to obtain the digital content (e.g., the corresponding subset of the digital content). In some instances, the agreement may allow access to new digital content upon creation. In some instances, a new agreement and/or a revision to the agreement may be prepared for new digital content in the content database 105 that may have been beyond the terms of an initial agreement. In these and other instances, the digital content in the content database 105 may be inaccessible without an agreement in place. For example, in instances in which a third subscriber may be interested in a portion of the digital content in the content database 105, but the third subscriber may not have an agreement with the content database 105, the third subscriber may be restricted access to the digital content in the content database 105.

In some embodiments, the first subscriber system 110 and or the second subscriber system 120 may be different practices, locations, groups, and/or any other differentiating factor. Each of the subscribers may be operable to obtain digital content from the content database 105 based on use with respect to the subscribers. For example, a first patient associated with the first subscriber system 110 may receive a first diagnosis. Further, the first patient may be located in a first room that may include the first display 115a and particular digital content associated with the first diagnosis may be provided to the first patient via the first display 115a. The first subscriber system 110 may have any number of rooms and/or displays, where each of the displays may be individually directed to display digital content associated with a diagnosis of a patient, where the patient may have viewing access of the display.

In some instances, the first room may be an individual room where a patient may receive a private consultation, a medical analysis, and/or a treatment, any of which may be individually delivered between the patient and a provider (e.g., a doctor, a physician assistant, a nurse, etc.). In such instances, the digital content displayed in the first room may be individually associated with the patient located therein, and may or may not include private information associated with the patient and/or a corresponding diagnosis.

Alternatively, or additionally, in some instances, the first room may be a group waiting room operable to allow multiple patients to wait for a diagnosis, consultation, and/or treatment, such as in an individual room. In instances in which the first room is a group waiting room, the digital content displayed therein may include general information associated with the first subscriber (e.g., the clinic/practice), the process that the multiple patients may experience at the first subscriber office, fee information, insurance information, and/or other general information associated with the first subscriber, doctors associated with the first subscriber, and so forth. In some instances, the digital content may include a general overview of multiple diagnoses (and/or potential diagnoses) of multiple patients that may be located in the group waiting room. For example, a display (e.g., the mth display 115b) may display general information (e.g., not patient specific detail) associated with a first diagnosis, a second diagnosis, and a third diagnosis corresponding to a first patient, a second patient, and/or a third patient while the first patient, the second patient, and the third patient wait in a waiting room associated with the mth display 115b.

In these and other instances, the digital content may be queued for display on any of the displays (e.g., the first display 115a, the mth display 115b, etc.) from one or more locations. The locations for determining the digital content to be displayed in a room associated with a particular patient may include a workstation within the room associated with the particular patient, a front desk workstation, one or more nurses workstations, a doctor's office workstation, and/or a remote workstation associated with the first subscriber system 110 (e.g., a doctor's remote device connected to the first subscriber system 110, such as via VPN). For example, upon admitting a first patient, a person at the front desk may direct digital content to be displayed associated with a past or current diagnosis on a display corresponding to a first room where the first patient is located. In another example, after determining a diagnosis for the first patient, a doctor may direct an assistant at a nurse's station to display digital content to the first patient corresponding to the determined diagnosis. In another example, after determining an initial diagnosis and confirming the diagnosis of the first patient in view of additional information (e.g., lab reports, similarity to other confirmed cases, etc.), the doctor may direct the digital content to be displayed to the first patient from a doctor's office workstation.

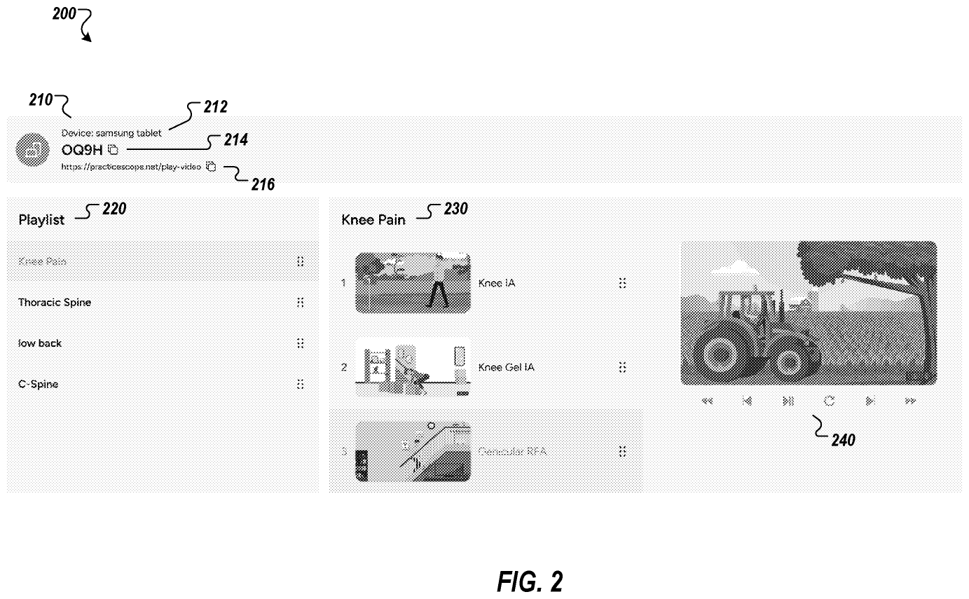

In some instances, the digital content may be displayed on one of the displays (e.g., the mth display 115b), which may include a tablet, laptop computer, smart phone, and/or other similar personal, portable electronic devices. The digital content displayed thereon may be directed by an entity, such as a receptionist, a nurse, and/or a doctor associated with the particular patient. For example, as illustrated in FIG. 2, a user interface 200 for information display for a patient may display a copy of a user interface being displayed to a particular patient.

The user interface 200 may include a device information portion 210 which may include a name of the display that the particular patient may be using to view the digital content. The device information portion 210 may include a display name 212, an access code 214, and/or a URL 216 that may be used to recreate the user interface 200 in a web browser. The user interface 200 may also include a playlist 220 that may be populated based on the profile associated with the particular patient. For example, the particular patient associated with the user interface 200 may have experienced knee pain, thoracic spine issues, lower back pain, and/or cervical spine issues, and the playlist 220 may be populated to include the same. As a patient selects one item from the playlist 220, a digital content window 230 may be displayed, which may include a subset of the digital content for viewing, where the subset may be associated with the selected playlist. In response to the particular patient selecting one of the subset in the digital content window 230, the corresponding digital content may be displayed in a display portion 240 of the user interface 200. As described herein, the display of the digital content, including the playlist 220, the digital content window 230, and/or the display portion 240 may be viewed by another entity, including as the patient may navigate to view various digital content.

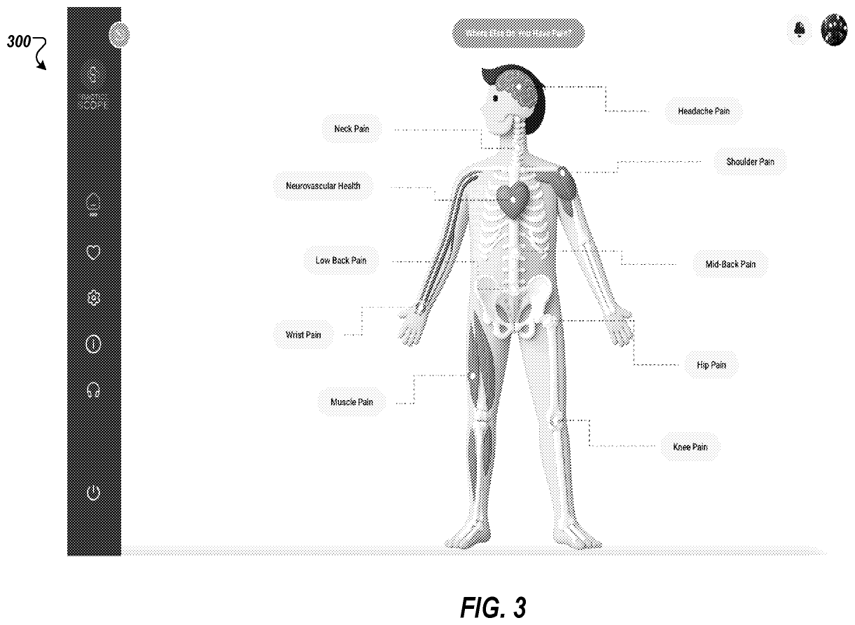

Alternatively, or additionally, the display may include an application that may be interacted with by the particular patient, where the application may operate on various operating systems (e.g., Android, iOS, etc.), such that the particular patient may direct the content to be displayed. For example, FIG. 3 illustrates an interactive information display 300 for a patient, where the particular patient may provide inputs to the interactive information display 300 to obtain particular digital content. For example, as illustrated in the interactive information display 300, a representative human body may be displayed along with common ailments that may be experienced by patients, and the particular patient may select one or more portions of the representative human body to have digital content displayed associated with the selection.

As such, the particular patient may use a user interface (e.g., such as the interactive information display 300) to obtain particular digital content corresponding to their particular condition (e.g., which may coincide with the profile or may differ from the profile). In these and other instances, the representative human body in the interactive information display 300 may illustrate various anatomic areas and/or corresponding selectable buttons that the particular patient may interact act with to obtain corresponding digital content. In instances in which the particular patient selects one of the anatomic areas and views the corresponding digital content, the interactive information display 300 may return to the overview screen and may allow the particular patient to view additional anatomic areas and/or corresponding digital content.

In some instances, there may be one digital content from the content database 105 that may correspond to one diagnosis. Alternatively, or additionally, in some instances, one digital content from the content database 105 may correspond to more than one diagnosis. Alternatively, or additionally, in some instances, more than one digital content from the content database 105 may correspond to one diagnosis. In these and other instances, the first subscriber system 110 (and/or entity associated with the first subscriber system 110) may direct any amount of digital content (that the first subscriber system 110 has access to from the content database 105) to a display where a first patient may view the digital content, based on a first diagnosis provided to the first patient.

In an example, a first patient may obtain a first diagnosis from a practitioner associated with the first subscriber system 110 and the first patient may be directed to a room containing a display, such as the first display 115a. The first subscriber system 110 may have obtained and/or may have access to particular digital content from the content database 105 that may correspond with the first diagnosis. The first subscriber system 110 may be operable to distribute digital content to any number of displays (e.g., the mth display 115b) for patients based on diagnoses provided to the patients.

In some instances, the implementation of the system 100, the content database 105, the first subscriber system 110, and/or the second subscriber system 120 may be on a local computing device, a cloud computing device, and/or a hybrid of local computing systems. For example, in a first implementation, the system 100 may be operated on a local computing system and in a second implementation, the system 100 may be operated on a multi-cloud system where portions of the system 100 may be disposed in different networked computing systems. Alternatively, or additionally, one or more components of the system 100 may be distributed. For example, the content database 105 may be disposed in a cloud computing device and the first subscriber system 110 and/or the second subscriber system 120 may individually be local computing devices.

Modifications, additions, or omissions may be made to the system 100 without departing from the scope of the present disclosure. For example, the designations of different elements in the manner described is meant to help explain concepts described herein and is not limiting. Further, the system 100 may include any number of other elements or may be implemented within other systems or contexts than those described. For example, any of the components of FIG. 1 may be divided into additional or combined into fewer components.

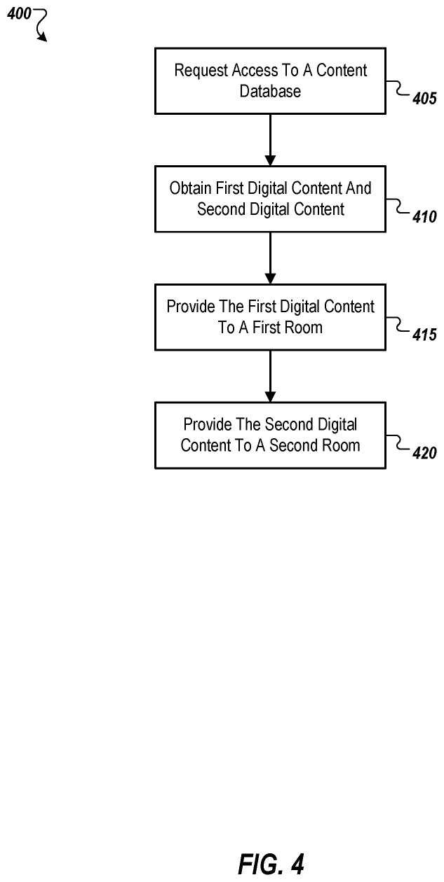

FIG. 4 illustrates a flowchart of an example method 400 of automated information display for patients. The method 400 may be performed by processing logic that may include hardware (circuitry, dedicated logic, etc.), software (such as is run on a general purpose computer system or a dedicated machine), or a combination of both, which processing logic may be included in any computer system or device such as the first subscriber system 110 or the second subscriber system 120 of FIG. 1.

For simplicity of explanation, methods described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification may be capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

At block 405, access to a content database may be requested. The content database may include a set of digital content. In some instances, the set of digital content may be associated with multiple diagnoses. In some instances, a portion of the content database may be inaccessible based on a level of access granted to a requestor.

At block 410, first digital content and second digital content may be obtained from the content database. The first digital content and the second digital content may be included in the set of digital content. In some instances, the first digital content may include one or more videos related to the first diagnosis, treatment of a condition associated with the first diagnosis, results of the treatment of the condition, and/or follow-up procedures associated with the treatment.

At block 415, the first digital content may be provided to a first room. In some instances, the first digital content may be provided to the first room based on an association between the first digital content and a first diagnosis given to a first patient.

At block 420, the second digital content may be provided to a second room. In some instances, the second digital content may be provided to the second room based on an association between the second digital content and a second diagnosis given to a second patient.

In some instances, particular digital content of the set of digital content may be associated with a first diagnosis of the multiple diagnoses. Alternatively, or additionally, more than one digital content of the set of digital content may be associated with a first medical procedure of the multiple diagnoses. Alternatively, or additionally, a particular digital content of the set of digital content may be associated with more than one diagnosis of the multiple diagnoses. In some instances, a request to add the third digital content to the content database may be submitted in response to a third digital content not being present in the content database.

Modifications, additions, or omissions may be made to the method 400 without departing from the scope of the present disclosure. For example, in some instances, the first digital content may be provided to a third room. The third digital content may be provided to the third room based on an association between the third digital content and the first diagnosis given to the third patient.

In another example, the designations of different elements in the manner described is meant to help explain concepts described herein and is not limiting. Further, the method 400 may include any number of other elements or may be implemented within other systems or contexts than those described.

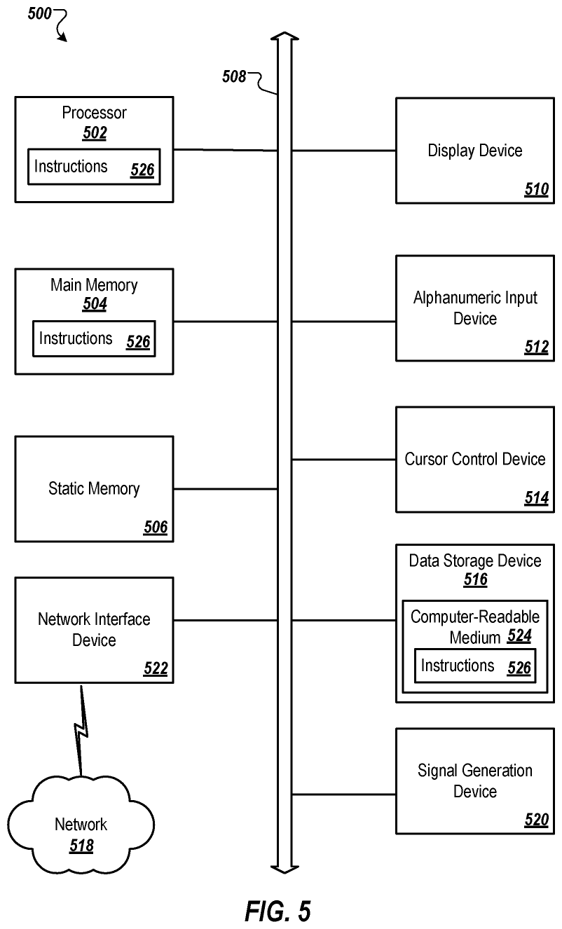

FIG. 5 illustrates an example computing device 500 within which a set of instructions for causing the machine to perform any one or more of the methods discussed herein may be executed. The computing device 500 may include a mobile phone, a smart phone, a netbook computer, a rackmount server, a router computer, a server computer, a personal computer, a mainframe computer, a laptop computer, a tablet computer, a desktop computer, or any computing device with at least one processor, etc., within which a set of instructions for causing the machine to perform any one or more of the methods discussed herein may be executed. In alternative implementations, the machine may be connected (e.g., networked) to other machines in a LAN, an intranet, an extranet, or the Internet. The machine may operate in the capacity of a server machine in client-server network environment. The machine may include a personal computer (PC), a set-top box (STB), a server, a network router, switch or bridge, or any machine capable of executing a set of instructions (sequential or otherwise) that specify actions to be taken by that machine. Further, while only a single machine is illustrated, the term “machine” may also include any collection of machines that individually or jointly execute a set (or multiple sets) of instructions to perform any one or more of the methods discussed herein.

The computing device 500 can include a processing device 502 (e.g., a processor), a main memory 504 (e.g., read-only memory (ROM), flash memory, dynamic random access memory (DRAM) such as synchronous DRAM (SDRAM)), a static memory 506 (e.g., flash memory, static random access memory (SRAM)) and a data storage device 516, which communicate with each other via a bus 508.

The processing device 502 represents one or more general-purpose processing devices such as a microprocessor, central processing unit, or the like. More particularly, the processing device 502 may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 502 may also include one or more special-purpose processing devices such as an application specific integrated circuit (ASIC), a field programmable gate array (FPGA), a digital signal processor (DSP), network processor, or the like. The processing device 502 is configured to execute instructions 526 for performing the operations and steps discussed herein.

The computing device 500 may further include a network interface device 522 which may communicate with a network 518. The computing device 500 also may include a display device 510 (e.g., a liquid crystal display (LCD) or a cathode ray tube (CRT)), an alphanumeric input device 512 (e.g., a keyboard), a cursor control device 514 (e.g., a mouse) and a signal generation device 520 (e.g., a speaker). In at least one implementation, the display device 510, the alphanumeric input device 512, and the cursor control device 514 may be combined into a single component or device (e.g., an LCD touch screen).

The data storage device 516 may include a computer-readable storage medium 524 on which is stored one or more sets of instructions 526 embodying any one or more of the methods or functions described herein. The instructions 526 may also reside, completely or at least partially, within the main memory 504 and/or within the processing device 502 during execution thereof by the computing device 500, the main memory 504 and the processing device 502 also constituting computer-readable media. The instructions may further be transmitted or received over a network 518 via the network interface device 522.

While the computer-readable storage medium 524 is shown in an example implementation to be a single medium, the term “computer-readable storage medium” may include a single medium or multiple media (e.g., a centralized or distributed database and/or associated caches and servers) that store the one or more sets of instructions. The term “computer-readable storage medium” may also include any medium that is capable of storing, encoding, or carrying a set of instructions for execution by the machine and that cause the machine to perform any one or more of the methods of the present disclosure. The term “computer-readable storage medium” may accordingly be taken to include, but not be limited to, solid-state memories, optical media and magnetic media.

A number of implementations have been described. Nevertheless, it will be understood that various modifications may be made without departing from the spirit and scope of the disclosure. Accordingly, other implementations are within the scope of the following claims.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, it is understood that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A method, comprising:
requesting access to a content database, the content database comprising a plurality of digital content;
obtaining first digital content and second digital content of the plurality of digital content from the content database;
providing the first digital content to a first room; and
providing the second digital content to a second room.

2. The method of claim 1, wherein:
the first digital content is provided to the first room based on an association between the first digital content and a first diagnosis given to a first patient; and
the second digital content is provided to the second room based on an association between the second digital content and a second diagnosis given to a second patient.

3. The method of claim 2, wherein the first digital content comprises one or more videos related to the first diagnosis, treatment of a condition associated with the first diagnosis, results of the treatment of the condition, and follow-up procedures associated with the treatment.

4. The method of claim 2, further comprising providing the first digital content to a third room, wherein the first digital content is provided to the third room based on an association between the first digital content and the first diagnosis given to a third patient.

5. The method of claim 2, wherein the first room is a waiting room for the first patient.

6. The method of claim 1, wherein the plurality of digital content is associated with a plurality of diagnoses.

7. The method of claim 6, wherein a particular digital content of the plurality of digital content is associated with a first diagnosis of the plurality of diagnoses.

8. The method of claim 6, wherein more than one digital content of the plurality of digital content is associated with a first medical procedure of the plurality of diagnoses.

9. The method of claim 6, wherein a particular digital content of the plurality of digital content is associated with more than one diagnosis of the plurality of diagnoses.

10. The method of claim 1, wherein a portion of the content database is inaccessible based on a level of access granted to a requestor.

11. The method of claim 1, wherein in response to a third digital content not being present in the content database, submitting a request to add the third digital content to the content database.

12. A system for automated information display, comprising:
at least one display associated with a room;
a content database including digital content comprising one or more videos and/or descriptions related to a procedure; and
a subscriber system operable to:
access a subset of the digital content in the content database;
obtain first digital content from the content database based on a first profile associated with a first patient; and
direct the first digital content to be displayed on the at least one display corresponding to the room where the first patient is located.

13. The system of claim 12, wherein the first profile comprises one or more of intake questions answered by the first patient, symptoms experienced by the first patient, a potential diagnosis associated with the first patient, and a diagnosis provided to the first patient.

14. The system of claim 12, wherein the digital content in the content database is arranged in one or more playlists, where a first playlist corresponds to a first diagnosis.

15. The system of claim 12, further comprising a second display associated with a second room, wherein the subscriber system is further operable to:
obtain second digital content from the content database based on a second profile associated with a second patient; and
direct the second digital content to be displayed on the second display corresponding to the second room where the second patient is located.

16. The system of claim 12, wherein the room is a private room where the first patient receives a private consultation, a medical analysis, or a treatment, and wherein the first digital content is individually associated with the first patient.

17. The system of claim 12, wherein the room is a group waiting room, and wherein the first digital content comprises one or more of general information associated with the subscriber system, a process associated with the subscriber system, fee information, insurance information, practitioners associated with the subscriber system, and various diagnoses that are not patient-specific.

18. The system of claim 12, wherein, in response to a particular diagnosis associated with the first patient not having corresponding digital content in the content database, the content database is operable to generate an indication that one or more digital content is to be created or added to correspond with the particular diagnosis.

19. The system of claim 12, wherein access to the content database is restricted such that the subscriber system has access to a first subset of the digital content in the content database and a second subscriber system has access to a second subset of the digital content in the content database.

20. The system of claim 12, wherein the first digital content comprises information related to a diagnosis associated with the first patient, treatment of a condition associated with the diagnosis, results of the treatment, or a follow-up procedure associated with the treatment.
